(19) United States
(12) Patent Application Publication
(10) Pub. No.: US 2021/0403412 A1
(43) Pub. Date: Dec. 30, 2021

(54) PRODUCTION OF NITROGEN-CONTAINING CHELATORS

(71) Applicant: Ascend Performance Materials Operations LLC, Houston, TX (US)

(72) Inventors: Zhensheng Ding, Houston, TX (US); Kyle Kissell, Houston, TX (US); Scott G. Moffatt, Houston, TX (US); Huayun Yu, Houston, TX (US)

(73) Assignee: Ascend Performance Materials Operations LLC, Houston, TX (US)

(21) Appl. No.: 17/363,770

(22) Filed: Jun. 30, 2021

Related U.S. Application Data
(60) Provisional application No. 63/046,245, filed on Jun. 30, 2020.

(51) Int. Cl. C07C 227/26; C07C 253/30

(52) U.S. Cl. CPC C07C 227/26; C07C 253/30

(57) ABSTRACT

Reaction pathways and conditions for the production of nitrogen-containing chelators, such as a glycine derivative, are described herein. In particular, the present disclosure describes a process for the production of a nitrile intermediate at a high yield and/or purity. This process includes reacting a dinitrile compound with an aldehyde and a hydrogen cyanide to form the nitrile intermediate. The nitrile intermediate may then be further processed to produce the chelators at a high yield and/or a high purity.

### CROSS REFERENCE TO RELATED APPLICATIONS

This application claims priority to U.S. Provisional Application No. 63/046,245, filed Jun. 30, 2020, which is incorporated herein by reference.

### FIELD

The present disclosure relates generally to the production of nitrogen-containing chelators. In particular, the present disclosure relates to reaction pathways and conditions for the production of nitrogen-containing chelators with high yield and/or purity.

### BACKGROUND

Chelators, also known as chelating agents, are organic compounds whose structures allow them to form bonds to a metal atom. Because chelators typically form two or more separate coordinate bonds to a single, central metal atom, chelators can be described as polydentate ligands. Chelators often include sulfur, nitrogen, and/or oxygen, which act as electron-donating atoms in bonds with the metal atom.

Chelators are useful in a variety of applications, where their propensity to form chelate complexes with metal atoms is important. Conventional uses of chelators include in nutritional supplements, in medical treatments (e.g., chelation therapy to remove toxic metals from the body), as contrast agents (e.g., in MRI scans), in domestic and/or industrial cleaners and/or detergents, in the manufacture of catalysts, in removal of metals during water treatment, and in fertilizers. For example, chelators play an important role in treatment of cadmium or mercury poisoning, because the chelators can be selected to selectively bind to the metals and facilitate excretion.

Conventional chelators include, for example, aminopolyphosphonates, polycarboxylates, ethylenediaminetetraacetic acid (EDTA), diethylenetriaminepentaacetic acid (DTPA), and nitrilotriacetic acid (NTA). These and other conventional chelators, however, exhibit a number of undesirable properties. Some conventional chelators do not demonstrate adequate activity or stability across a wide pH and/or temperature range. Some conventional chelators exhibit an unacceptably high toxicity. Some conventional chelators do not exhibit adequate solubility in aqueous and/or organic solvents. Some conventional chelators have low biodegradability and present high environmental risk.

Glycine derivatives, such as alanine-N,N-diacetonitrile, are a class of chelators that may exhibit these desirable properties. These chelators, which may be structural derivatives of the amino acid glycine, exhibit sufficient (or improved) activity and/or stability across a wide pH and/or temperature range, low toxicity, adequate solubility, and/or high biodegradability. Unfortunately, conventional processes, such as Strecker amino acid synthesis, for preparing glycine-derivative chelators are typically inefficient.

Thus, the need exists for processes for producing nitrile chelators and intermediates used to produce the nitrile chelators that demonstrate both efficiency and cost-effectiveness improvements. In particular, the need exists for producing glycine-derivative nitrile chelators without the need for a separate crystallization step. The resultant nitrile chelators and intermediates should have adequate stability and activity across a wide pH and/or temperature range, low toxicity, and suitable biodegradability.

### SUMMARY

In some aspects, the present disclosure relates to a process for preparing a nitrile intermediate, the process comprising: reacting a dinitrile compound with a hydrogen cyanide and an aldehyde of the formula R—CHO, where R is (C₁-C₁₀)alkyl, (C₁-C₁₀)haloalkyl, (C₁-C₁₀)alkenyl, or (C₁-C₁₀)alkyl carboxylate, in an aqueous solution to form the nitrile intermediate; wherein a nitrile intermediate seed is employed during the reaction. In some cases, the reacting forms the nitrile intermediate as a crystalline solid. In some cases, the process does not comprise a crystallization step. In some cases, the dinitrile compound has a chemical structure:

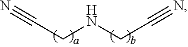

wherein a is from 0 to 5 and b is from 0 to 5. In some cases, the nitrile intermediate is an alanine-N,N-dinitrile. In some cases, the reacting does not form the nitrile intermediate as an emulsion. In some cases, the nitrile intermediate is formed at a yield greater than 70%. In some cases, the reacting comprises providing a dinitrile compound solution comprising the dinitrile compound In some cases, the reacting comprises heating the dinitrile compound solution to a temperature from 25° C. to 50° C. In some cases, the reacting comprises adjusting the pH of the dinitrile compound solution to a pH ranging from 0.5 to 4.0. In some cases, the reacting comprises adjusting the pH of a reaction mixture to a pH ranging from 0.5 to 4.0. In some cases, the reacting comprises adding the nitrile intermediate seed to a reaction mixture. In some cases, the reacting comprises heating a reaction mixture to a temperature from 30° C. to 80° C. In some cases, the reacting comprises maintaining the reaction mixture at the temperature for a period of time from 30 minutes to 180 minutes. In some cases, the reacting comprises: providing a dinitrile compound solution comprising the dinitrile compound; adjusting the pH of the dinitrile compound solution to a pH ranging from 0.5 to 4.0; heating the dinitrile compound solution; adding the aldehyde to the heated dinitrile compound solution to form a first intermediate solution; adding the nitrile intermediate seed to the first intermediate solution to form a second intermediate solution; adding hydrogen cyanide to the second intermediate solution to form a third intermediate solution; and heating the third intermediate solution to form the nitrile intermediate. In some cases, the pH of the reaction mixture is reduced by at least 2.0, optionally by adding sulfuric acid. In some cases, the reacting is carried out at a pH less than 5.0. In some cases, the third intermediate solution is heated at a rate from 1° C./hour to 60° C./hour. In some cases, the amount of nitrile intermediate seed added is less than 1% the theoretical yield of the nitrile intermediate. In some cases, the second intermediate solution comprises from 0.001 wt. % to 1 wt. % nitrile intermediate seed. In some cases, hydrogen cyanide is added at a rate less than 1 g/minute. In some cases, the nitrile intermediate is formed at a yield greater than 90%. In some cases, the reacting is carried out at a temperature from 25° C. to 40° C.

In some aspects, the process further comprises forming a glycine derivative from the nitrile intermediate. In some cases, the glycine derivative has a chemical structure:

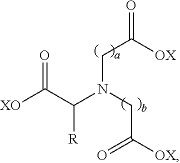

wherein: R is (C₁-C₁₀)alkyl, (C₁-C₁₀)haloalkyl, (C₁-C₁₀)alkenyl, or (C₁-C₁₀)alkyl carboxylate, X is hydrogen, an alkali metal, an alkaline earth metal, or ammonium, a is from 0 to 5, and b is from 0 to 5. In some cases, R is (C₁-C₅)alkyl, a is from 1 to 3, and/or b is from 1 to 3. In some cases, the forming the glycine derivative comprises hydrolyzing the nitrile intermediate. In some cases, the hydrolyzing comprises reacting the nitrile intermediate with an inorganic hydroxide selected from the group consisting of ammonium hydroxide, calcium hydroxide, lithium hydroxide, magnesium hydroxide, potassium hydroxide, sodium hydroxide, and combinations thereof. In some cases, the glycine derivative is alanine-N,N-diacetic acid. In some cases, the glycine derivative is formed at a yield of at least 60%.

### DETAILED DESCRIPTION

### Introduction

As noted, the present disclosure describes reaction pathways and conditions for the production of nitrogen-containing chelator intermediates and the chelators, e.g., glycine derivatives, produced therefrom. In particular, the present disclosure describes a reaction scheme for the efficient production of a nitrile intermediate at a high yield and/or purity. The nitrile intermediate may then be further processed to produce the chelators at a high yield and/or a high purity.

The reaction pathways and conditions described herein advantageously produce the nitrile intermediate in crystalline form (crystalline solid), e.g., without the need for a separate crystallization step. Conventional processes such as Strecker amino acid synthesis, in contrast, are inefficient and produce a nitrile intermediate in non-crystalline form, e.g., as an emulsion, which then requires an inefficient crystallization step. This is typically accomplished by complicated mechanical means, such as complex agitation procedures. The crystallization step reduces the efficiency of the overall reaction and provides further opportunity for the loss of product and/or the formation of impurities. The elimination of the need for crystallizing beneficially increases the efficiency of the reaction. For example, without the need for a separate crystallization step, the nitrile intermediate can be produced and collected more quickly. The crystalline nitrile intermediate also better facilitates conversion to the nitrogen-containing chelator. In addition, removing the crystallization step reduces the costs associated with the production of the nitrogen-containing chelators.

The present disclosure describes a process for preparing a nitrile intermediate by reacting a dinitrile compound with a hydrogen cyanide and an aldehyde (in an aqueous solution). In this reaction, the dinitrile may have the structure:

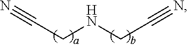

wherein a is from 0 to 5 and b is from 0 to 5, and the aldehyde may have the formula R—CHO, where R is (C₁-C₁₀)alkyl, (C₁-C₁₀)haloalkyl, (C₁-C₁₀)alkenyl, or (C₁-C₁₀)alkyl carboxylate.

In this process, a nitrile intermediate seed is employed during the reaction. For example, the seed is added to one or more of the (intermediate) reaction mixtures of the reacting step and reaction sub-steps. The nitrile intermediate seed, employed as disclosed herein, has been found to beneficially promote the formation of the nitrile intermediate in crystalline form (i.e. as a crystalline solid). As a result, the (crystalline) nitrile intermediate is surprisingly produced with high purity and/or high yield. Conventional processes do not employ nitrile intermediate seeds, and, as such, require significant additional processing to achieve crystallization, e.g., controlled agitation to produce crystals.

As discussed in detail below, the reacting of the dinitrile compound, hydrogen cyanide, and aldehyde to form the nitrile intermediate may take many forms. The reacting may comprise combining the reactants in an aqueous solution and allowing the reaction to proceed. In some cases, the reactants are combined substantially simultaneously. In some cases, the reactants are combined in particular order.

In some cases, the reacting comprises controlling the addition of the reactants (including the seed) as well as the reaction conditions. For example, in some embodiments, the various reactants may be added and/or combined in a specific order, and the nitrile intermediate seed may be added at specific points in the overall reaction scheme. Controlling the reaction according to the present disclosure may provide for increased yield and/or purity of the nitrile intermediate.

### Reactants

### Dinitrile Compound

According to the present disclosure, a nitrile intermediate is produced from a dinitrile compound (as a reactant). The structure of the dinitrile compound is not particularly limited, and any organic compound having at least two nitrile, or cyano (—C≡N), functional groups may be used. For example, the dinitrile compound may comprise a saturated or unsaturated carbon chain having two or more nitrile functional groups. In some embodiments, the nitrile functional groups may be moieties of a carbon chain that comprises one or more heteroatoms, such as oxygen, nitrogen, sulfur, or phosphorus.

In some embodiments, the dinitrile compound is an organic compound having two nitrile functional groups as moieties on a carbon chain that includes a nitrogen heteroatom. For example, the dinitrile compound may have a chemical structure:

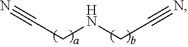

wherein a is from 0 to 5 and b is from 0 to 5. In some embodiments, the dinitrile compound may have the above chemical structure, wherein a is 1, and b is 0, 1, 2, 3, 4, or 5. In some embodiments, the dinitrile compound may have the above chemical structure, wherein a is 1 or 2, and be is 0, 1, 2, 3, or 4. In some embodiments, the dinitrile compound may have the above chemical structure, wherein a is 1, 2, or 3, and b is 1, 2, or 3. Exemplary dinitrile compounds according to the above chemical structure include ((cyanomethyl)amino)acetonitrile, ((cyanomethyl)amino)propanenitrile, ((cyanomethyl)amino)butanenitrile, ((cyanomethyl)amino)pentanenitrile, ((cyanoethyl)amino)acetonitrile, ((cyanoethyl)amino)propanenitrile, ((cyanoethyl)amino)butanenitrile, ((cyanoethyl)amino)pentanenitrile, ((cyanopropyl)amino)acetonitrile, ((cyanopropyl)amino)propanenitrile, ((cyanopropyl)amino)butanenitrile, ((cyanopropyl)amino)pentanenitrile, ((cyanobutyl)amino)acetonitrile, ((cyanobutyl)amino)propanenitrile, ((cyanobutyl)amino)butanenitrile, ((cyanobutyl)amino)pentanenitrile, ((cyanopropyl)amino)acetonitrile, ((cyanopropyl)amino)propanenitrile, ((cyanopropyl)amino)butanenitrile, and ((cyanopropyl)amino)pentanenitrile.

In some embodiments, the dinitrile compound may be dissolved in a solution, e.g., the dinitrile compound may be a component of a dinitrile compound solution (discussed in detail below). For example, the dinitrile compound may be mixed with and/or dissolved in a solvent. The composition of the dinitrile compound solution is not particularly limited and may be any solution of the dinitrile compound. In some embodiments, for example, the dinitrile compound solution may comprise the dinitrile compound dissolved in an aqueous solvent, e.g., water, an organic solvent, or a solvent system of both aqueous and organic solvents.

### Aldehyde

The aldehyde may vary widely and many suitable aldehydes are known. In particular, the aldehyde may have a chemical formula R—CHO, where R is (C₁-C₁₀)alkyl, (C₁-C₁₀)haloalkyl, (C₁-C₁₀)alkenyl, or (C₁-C₁₀)alkyl carboxylate. In some embodiments, R of the aldehyde is (C₁-C₁₀)alkyl, e.g., (C₁-C₉)alkyl, (C₁-C₈)alkyl, (C₁-C₇)alkyl, (C₁-C₆)alkyl, or (C₁-C₅)alkyl. In some embodiments, R of the aldehyde is (C₁-C₁₀)haloalkyl, e.g., (C₁-C₉)haloalkyl, (C₁-C₈)haloalkyl, (C₁-C₇)haloalkyl, (C₁-C₆)haloalkyl, or (C₁-C₅)haloalkyl. In some embodiments, R of the aldehyde is (C₁-C₁₀)alkenyl, e.g., (C₂-C₁₀)alkenyl, (C₁-C₉)alkenyl, (C₂-C₉)alkenyl, (C₁-C₈)alkenyl, (C₂-C₈)alkenyl, (C₁-C₇)alkenyl, (C₂-C₇)alkenyl, (C₁-C₆)alkenyl, (C₂-C₆)alkenyl, (C₁-C₅)alkenyl or (C₂-05)alkenyl. In some embodiments, R of the aldehyde is (C₁-C₁₀)alkyl carboxylate, e.g., (C₁-C₉)alkyl carboxylate, (C₁-C₈)alkyl carboxylate, (C₁-C₇)alkyl carboxylate, (C₁-C₆)alkyl carboxylate, or (C₁-C₅)alkyl carboxylate. For example, the aldehyde may comprise a saturated or unsaturated, straight or branched carbon chain, e.g., a terminal carbonyl functional group. Exemplary aldehydes include acetaldehyde, propionaldehyde, butyraldehyde, pentanal, propenal, butenal, formyl ethanoic acid, formyl propionic acid, and formyl butanoic acid.

As noted above, the order of the addition of the aldehyde to the other reactants may vary widely. In some cases, an aldehyde is added to and/or reacted with the dinitrile compound (optionally in a heated dinitrile compound solution) to form a first intermediate solution. For example, the aldehyde may be added to the dinitrile compound solution, and/or the dinitrile compound may be added to aldehyde. In some cases, the aldehyde may be added to a solution comprising the dinitrile compound and the hydrogen cyanide.

In some embodiments, the nitrile intermediate seed is added to and/or reacted with the first intermediate solution to form a second intermediate solution. As noted, the nitrile intermediate seed may be added at other points in the reaction scheme, examples of which are discussed in more detail herein.

The amount of aldehyde used in the reaction, e.g., the amount aldehyde present in the first intermediate solution or the second intermediate solution, is not particularly limited. The amount of aldehyde used may be based on the amount of dinitrile compound. In some embodiments, for example, an amount of aldehyde is added such that the molar ratio of the aldehyde to the dinitrile compound is from 0.1:1 to 10:1, e.g., from 0.1:1 to 8:1, from 0.1:1 to 6:1, from 0.1:1 to 4:1, from 0.1:1 to 2:1, from 0.2:1 to 10:1, from 0.2:1 to 8:1, from 0.2:1 to 6:1, from 0.2:1 to 4:1, from 0.2:1 to 2:1, from 0.4:1 to 10:1, from 0.4:1 to 8:1, from 0.4:1 to 6:1, from 0.4:1 to 4:1, from 0.4:1 to 2:1, from 0.5:1 to 10:1, from 0.5:1 to 8:1, from 0.5:1 to 6:1, from 0.5:1 to 4:1, from 0.5:1 to 2:1, from 0.8:1 to 10:1, from 0.8:1 to 8:1, from 0.8:1 to 6:1, from 0.8:1 to 4:1, or from 0.8:1 to 2:1. In terms of lower limits, the molar ratio of the aldehyde to the dinitrile compound may be greater than 0.1:1, e.g., greater than 0.2:1, greater than 0.4:1, greater than 0.5:1, or greater than 0.8:1. In terms of upper limits, the molar ratio of the aldehyde to the dinitrile compound may be less than 10:1, e.g., less than 8:1, less than 6:1, less than 4:1, or less than 2:1.

### Hydrogen Cyanide

The hydrogen cyanide (HCN) is added to and/or reacted with one or more of the other reactants. The hydrogen cyanide may be combined with the dinitrile compound before or after the aldehyde is introduced. In some embodiments, the aldehyde and the hydrogen cyanide are combined with the dinitrile compound at substantially the same time, e.g., simultaneously or within several minutes of each other. In some embodiments, the hydrogen cyanide is combined with the first intermediate solution, e.g., comprising the dinitrile compound and the aldehyde without the nitrile intermediate seed, or the second intermediate solution, e.g., comprising the dinitrile compound, the aldehyde, and the nitrile intermediate seed. For example, the hydrogen cyanide may be added to the first intermediate solution, and/or the first intermediate solution may be added to the dinitrile compound solution. In some embodiments, for example, the hydrogen cyanide may be added to the second intermediate solution (discussed below) to produce the third intermediate solution. In some embodiments, the hydrogen cyanide is added to and/or reacted with the dinitrile compound solution.

The HCN, in some instances, may be employed in the form of a solution comprising HCN and the solution may be reacted with the dinitrile compound (and the aldehyde) as described herein.

The amount of hydrogen cyanide used in the reaction, e.g., the amount hydrogen cyanide added to the dinitrile compound solution, is not particularly limited. The amount of hydrogen cyanide used may be based on the amount of dinitrile compound. In some embodiments, for example, an amount of hydrogen cyanide is added such that the molar ratio of the hydrogen cyanide to the dinitrile compound is from 0.1:1 to 10:1, e.g., from 0.1:1 to 8:1, from 0.1:1 to 6:1, from 0.1:1 to 4:1, from 0.1:1 to 2:1, from 0.2:1 to 10:1, from 0.2:1 to 8:1, from 0.2:1 to 6:1, from 0.2:1 to 4:1, from 0.2:1 to 2:1, from 0.4:1 to 10:1, from 0.4:1 to 8:1, from 0.4:1 to 6:1, from 0.4:1 to 4:1, from 0.4:1 to 2:1, from 0.5:1 to 10:1, from 0.5:1 to 8:1, from 0.5:1 to 6:1, from 0.5:1 to 4:1, from 0.5:1 to 2:1, from 0.8:1 to 10:1, from 0.8:1 to 8:1, from 0.8:1 to 6:1, from 0.8:1 to 4:1, or from 0.8:1 to 2:1. In terms of lower limits, the molar ratio of the hydrogen cyanide to the dinitrile compound may be greater than 0.1:1, e.g., greater than 0.2:1, greater than 0.4:1, greater than 0.5:1, or greater than 0.8:1. In terms of upper limits, the molar ratio of the hydrogen cyanide to the dinitrile compound may be less than 10:1, e.g., less than 8:1, less than 6:1, less than 4:1, or less than 2:1.

### Nitrite Intermediate Seed

As noted above, the disclosed processes employ a nitrile intermediate seed during the reaction. The timing of the addition of the nitrile intermediate seed to one or more of the reaction mixtures may vary. The addition of the nitrile intermediate seed has surprisingly been found to greatly improve the preparation of the nitrile intermediate, and subsequently the glycine derivative. In particular, the addition of the nitrile intermediate seed provides for the formation of the nitrile intermediate, e.g., by the reaction of the dinitrile compound, the aldehyde, and the hydrogen cyanide, in crystalline form. Said another way, in some cases, the processes described herein produce crystalline nitrile intermediate due to the addition of the nitrile intermediate seed during the reaction. Furthermore, the formation of crystals in situ during the reaction contributes to improved yield and purity of the nitrile intermediate produced by the reaction.

Generally, the nitrile intermediate seed is an organic compound having at least two nitrile, or cyano, functional groups and at least one carboxyl functional group. Exemplary nitrile intermediates include alanine-N,N-diacetonitrile, alanine-N,N-dipropionitrile, alanine-N,N-dibutyronitrile, alanine-N-acetonitrile-N-propionitrile, alanine-N-acetonitrile-N-butyronitrile, ethyl glycine-N,N-diacetonitrile, ethyl glycine-N,N-dipropionitrile, ethyl glycine-N,N-dibutyronitrile, ethyl glycine-N-acetonitrile-N-propionitrile, ethyl glycine-N-acetonitrile-N-butyronitrile, propyl glycine-N,N-diacetonitrile, propyl glycine-N,N-dipropionitrile, propyl glycine-N,N-dibutyronitrile, propyl glycine-N-acetonitrile-N-propionitrile, and propyl glycine-N-acetonitrile-N-butyronitrile.

In some embodiments, the chemical composition of the nitrile intermediate seed may be defined in relation to the nitrile intermediate to be formed by the reaction. For example, the nitrile intermediate seed may comprise substantially the same chemical structure, e.g., the same or slightly modified chemical structure, as the nitrile intermediate. Thus, any composition of the nitrile intermediate (discussed in detail below) may be used as the nitrile intermediate seed. The nitrile intermediate seed may be solid of the nitrile intermediate or may be a liquid solution comprising the nitrile intermediate. In some embodiments, for example, the nitrile intermediate seed is a solid crystal of the nitrile intermediate.

In some of the processes described herein, the nitrile intermediate seed is added to a reaction mixture before and/or during the reaction step. In some embodiments, the nitrile intermediate seed may combined with the dinitrile compound, e.g., the nitrile intermediate seed may be added to the dinitrile compound solution before the addition of both the aldehyde and the hydrogen cyanide. In some embodiments, the nitrile intermediate seed is added to the reaction mixture after the addition of the aldehyde (and before addition of the hydrogen cyanide). For example, the nitrile intermediate seed may be combined with the first intermediate solution, e.g., comprising the dinitrile compound and the aldehyde, to produce a second intermediate solution. In some embodiments, the nitrile intermediate seed is added to the reaction mixture after the addition of the hydrogen cyanide. In some embodiments, the nitrile intermediate seed is added to the reaction mixture at substantially the same time as the aldehyde and/or the hydrogen cyanide.

In some cases, only a small amount of the nitrile intermediate seed is required to produce the effects described herein, but larger amounts are contemplated. The amount of nitrile intermediate seed added to the reaction mixture may be described by reference to the theoretical yield of the nitrile intermediate by the reaction. In some embodiments, the amount of nitrile intermediate seed added to the reaction mixture is less than 1% of the theoretical yield of the nitrile intermediate by the reaction, e.g., less than 0.8%, less than 0.5%, less than 0.2%, less than 0.1%, or less than 0.08%. In terms of lower limits, the amount of nitrile intermediate added to the reaction mixture is greater than 0.0001% the theoretical yield of the nitrile intermediate by the reaction, e.g., greater than 0.0005%, greater than 0.001%, greater than 0.005%, or greater than 0.008%.

The amount of nitrile intermediate seed added to the reaction mixture may also be described by reference to the weight percentage of the nitrile intermediate seed in the reaction mixture, e.g., the weight percentage of the nitrile intermediate seed in the second intermediate solution. In some embodiments, the second intermediate solution comprises from 0.001 wt. % to 1 wt. % of the nitrile intermediate seed, e.g., from 0.001 wt. % to 0.5 wt. %, from 0.001 wt. % to 0.1 wt. %, from 0.001 wt. % to 0.1 wt. %, from 0.001 wt. % to 0.08 wt. %, from 0.005 wt. % to 1 wt. %, from 0.005 wt. % to 0.5 wt. %, from 0.005 wt. % to 0.1 wt. %, from 0.005 wt. % to 0.1 wt. %, from 0.005 wt. % to 0.08 wt. %, from 0.008 wt. % to 1 wt. %, from 0.008 wt. % to 0.5 wt. %, from 0.008 wt. % to 0.1 wt. %, from 0.008 wt. % to 0.1 wt. %, from 0.008 wt. % to 0.08 wt. %, from 0.01 wt. % to 1 wt. %, from 0.01 wt. % to 0.5 wt. %, from 0.01 wt. % to 0.1 wt. %, from 0.01 wt. % to 0.1 wt. %, or from 0.01 wt. % to 0.08 wt. %. In terms of upper limits, the second intermediate solution may comprise less than 1 wt. % nitrile intermediate seed, e.g., less than 0.5 wt. %, less than 0.1 wt. %, or less than 0.08 wt. %.

### Reaction

As noted above, the reacting step of the processes described herein may comprise controlling the addition of the reactants as well as the reaction conditions. For example, in some embodiments, the various reactants may be added and/or combined in a specific order, and the nitrile intermediate seed may be added at specific points in the overall reaction scheme. The reaction conditions described herein may further improve the production of the nitrile intermediate, e.g., the purity and/or yield of the nitrile intermediate. In particular, the present disclosure provides temperature and pH conditions, which the present inventors have surprisingly found produce the purity and/or the yield of the nitrile intermediate by the reaction described herein.

Controlling the reaction according to the present disclosure may provide for increased yield and/or purity of the nitrile intermediate.

### Individual Components

In some embodiments, the reacting comprises providing a dinitrile compound solution comprising the dinitrile compound. For example, the process may include dissolving the dinitrile compound in a solvent to prepare the dinitrile compound solution. The composition of the dinitrile compound solution is not particularly limited and may be any solution of the dinitrile compound. In some embodiments, for example, the dinitrile compound solution may comprise the dinitrile compound dissolved in an aqueous solvent, e.g., water. In some embodiments, the dinitrile compound solution may comprise the dinitrile compound dissolved in an organic solvent. In some embodiments, the dinitrile compound solution is a solution of the dinitrile dissolved in a solvent system of both aqueous and organic solvents.

The concentration of the dinitrile compound solution is not particularly limited. In some embodiments, the dinitrile compound solution comprises from 5 wt. % to 60 wt. % of the dinitrile compound, e.g., from 5 wt. % to 55 wt. %, from 5 wt. % to 50 wt. %, from 5 wt. % to 45 wt. %, from 5 wt. % to 40 wt. %, from 10 wt. % to 60 wt. %, from 10 wt. % to 55 wt. %, from 10 wt. % to 50 wt. %, from 10 wt. % to 45 wt. %, from 10 wt. % to 40 wt. %, from 15 wt. % to 60 wt. %, from 15 wt. % to 55 wt. %, from 15 wt. % to 50 wt. %, from 15 wt. % to 45 wt. %, from 15 wt. % to 40 wt. %, from 20 wt. % to 60 wt. %, from 20 wt. % to 55 wt. %, from 20 wt. % to 50 wt. %, from 20 wt. % to 45 wt. %, or from 20 wt. % to 40 wt. %. In terms of lower limits, the dinitrile compound solution may comprise greater than 5 wt. % of the dinitrile compound, e.g., greater than 10 wt. %, greater than 15 wt. %, or greater than 20 wt. %. In terms of upper limits, the dinitrile compound solution may comprise less than 60 wt. % of the dinitrile compound, e.g., less than 55 wt. %, less than 50 wt. %, less than 45 wt. %, or less than 40 wt. %.

Without being limited by theory, the dinitrile compound solution may be provided for the reaction at any temperature or may be heated to a target temperature. In some embodiments, the dinitrile compound solution is provided at room temperature. In some embodiments, the dinitrile compound is from about 10° C. to about 30° C., e.g., from about 10° C. to about 29° C., from about 10° C. to about 28° C., from about 10° C. to about 27° C., from about 10° C. to about 26° C., from about 10° C. to about 25° C., from about 12° C. to about 30° C., from about 12° C. to about 29° C., from about 12° C. to about 28° C., from about 12° C. to about 27° C., from about 12° C. to about 26° C., from about 12° C. to about 25° C., from about 14° C. to about 30° C., from about 14° C. to about 29° C., from about 14° C. to about 28° C., from about 14° C. to about 27° C., from about 14° C. to about 26° C., from about 14° C. to about 25° C., from about 18° C. to about 30° C., from about 18° C. to about 29° C., from about 18° C. to about 28° C., from about 18° C. to about 27° C., from about 18° C. to about 26° C., from about 18° C. to about 25° C., from about 20° C. to about 30° C., from about 20° C. to about 29° C., from about 20° C. to about 28° C., from about 20° C. to about 27° C., from about 20° C. to about 26° C., from about 20° C. to about 25° C., from about 22° C. to about 30° C., from about 22° C. to about 29° C., from about 22° C. to about 28° C., from about 22° C. to about 27° C., from about 22° C. to about 26° C., or from about 22° C. to about 25° C.

In some cases, the reacting comprises adjusting the temperature of the dinitrile compound solution. In some embodiments, the dinitrile compound solution is heated, e.g., before combination with the aldehyde and the hydrogen cyanide, to a temperature from 25° C. and 50° C., e.g., from 25° C. to 48° C., from 25° C. to 45° C., from 25° C. to 42° C., from 25° C. to 40° C., from 26° C. and 50° C., from 26° C. to 48° C., from 26° C. to 45° C., from 26° C. to 42° C., from 26° C. to 40° C., from 27° C. and 50° C., from 27° C. to 48° C., from 27° C. to 45° C., from 27° C. to 42° C., from 27° C. to 40° C., from 28° C. and 50° C., from 28° C. to 48° C., from 28° C. to 45° C., from 28° C. to 42° C., from 28° C. to 40° C., from 29° C. and 50° C., from 29° C. to 48° C., from 29° C. to 45° C., from 29° C. to 42° C., from 29° C. to 40° C., from 30° C. and 50° C., from 30° C. to 48° C., from 30° C. to 45° C., from 30° C. to 42° C., or from 30° C. to 40° C. In terms of lower limits, the dinitrile compound solution may be heated to a temperature greater than 25° C., e.g., greater than 26° C., greater than 27° C., greater than 28° C., greater than 29° C., or greater than 30° C. In terms of upper limits, the dinitrile compound solution may be heated to a temperature less than 50° C., e.g. less than 48° C., less than 45° C., less than 42° C., or less than 40° C.

The acidity and/or alkalinity of the reactants (and/or the reaction mixture and/or the various intermediate mixtures) can greatly affect the progress of the reaction. In particular, the reaction of the present disclosure may require an acidic environment, e.g., pH less than 7.

In some embodiments, the dinitrile compound solution is prepared at an approximately neutral pH, e.g., a pH ranging from 5 to 9, e.g., from 6 to 8, from 6.5 to 7.5, or from 6.75 to 7.25. Thus, in some cases, the reacting comprises modifying, e.g., controlling and/or adjusting, the pH of the dinitrile compound solution. In some embodiments, the pH can be modified by the addition of a mineral acid, e.g., hydrochloric acid, nitric acid, phosphoric acid, sulfuric acid, boric acid, hydrofluoric acid, hydrobromic acid, perchloric acid, or hydriodic acid.

In some embodiments, the dinitrile compound solution is adjusted to a pH ranging from 0.5 to 4, e.g., from 0.5 to 3.8, from 0.5 to 3.6, from 0.5 to 3.4, from 0.5 to 3.2, from 0.5 to 3.0, from 0.8 to 4, from 0.8 to 3.8, from 0.8 to 3.6, from 0.8 to 3.4, from 0.8 to 3.2, from 0.8 to 3.0, from 1.0 to 4, from 1.0 to 3.8, from 1.0 to 3.6, from 1.0 to 3.4, from 1.0 to 3.2, from 1.0 to 3.0, from 1.2 to 4, from 1.2 to 3.8, from 1.2 to 3.6, from 1.2 to 3.4, from 1.2 to 3.2, from 1.2 to 3.0, from 1.5 to 4, from 1.5 to 3.8, from 1.5 to 3.6, from 1.5 to 3.4, from 1.5 to 3.2, or from 1.5 to 3.0.

In some cases, the reacting comprises adjusting the temperature of the aldehyde (or of the solution containing the aldehyde). In some embodiments, the aldehyde is heated or chilled, e.g., before combination with the dinitrile compound solution, to a temperature from 1° C. to 40° C., e.g., from 2° C. to 35° C., from 3° C. to 30° C., or from 4° C. to 25° C. In some cases, the reacting comprises modifying, e.g., controlling and/or adjusting, the pH of the aldehyde, e.g., before combination with the dinitrile compound solution, to a pH from 0.5 to 9.

As noted, the reacting may comprise adding the aldehyde (or a solution containing the aldehyde). The method of adding the aldehyde is not particularly limited. In some cases, for example, the aldehyde may be added to the dinitrile compound solution by a syringe, e.g., a sub-surface syringe. In one embodiment, the aldehyde is added at a rate from 5 g/min to 25 g/min, e.g., from 8 g/min to 22 g/min, from 10 g/min to 20 g/min, or from 12 g/min to 15 g/min. In terms of lower limits, the addition rate may be greater than 5 g/min, e.g., greater than 8 g/min, greater than 10 g/min, or greater than 12 g/min. In terms of upper limits, the addition rate may be less than 25 g/min, e.g., less than 22 g/min, less than 20 g/min, less than 18 g/min, or less than 15 g/min.

In some cases, the reacting comprises adjusting the temperature of the hydrogen cyanide (or the solution containing the hydrogen cyanide). In some embodiments, the hydrogen cyanide is heated or chilled, e.g., before combination with the dinitrile compound solution, to a temperature from 1° C. to 40° C., e.g., from 2° C. to 35° C., from 3° C. to 30° C., or from 4° C. to 25° C. In some cases, the reacting comprises modifying, e.g., controlling and/or adjusting, the pH of the hydrogen cyanide, e.g., before combination with the dinitrile compound solution, to a pH from 0.5 to 9.

As noted, the reacting may comprise adding the hydrogen cyanide (or a solution containing the hydrogen cyanide). The method of adding the hydrogen cyanide is not particularly limited. In some cases, for example, the hydrogen cyanide may be added to the dinitrile compound solution by a syringe, e.g., a sub-surface syringe. In one embodiment, the hydrogen cyanide is added at a rate from 0.01 g/min to 1 g/min, e.g., from 0.02 g/min to 0.5 g/min, from 0.05 g/min to 0.3 g/min, or from 0.08 g/min to 0.2 g/min. In terms of lower limits, the addition rate may be greater than 0.01 g/min, e.g., greater than 0.02 g/min, greater than 0.05 g/min, or greater than 0.08 g/min. In terms of upper limits, the addition rate may be less than 1 g/min, e.g., less than 0.5 g/min, less than 0.3 g/min, or less than 0.2 g/min.

### Reaction Conditions

In some cases, reacting comprises combining the dinitrile compound, e.g., the dinitrile compound solution, the aldehyde, the hydrogen cyanide, and/or the nitrile intermediate seed. In some embodiments, all reactants are combined simultaneously or substantially simultaneously, e.g., within a few minutes of each other. In some embodiments, the reactants are combined in a particular order. In some embodiments, for example, the dinitrile compound, e.g., the dinitrile compound solution, and the aldehyde may be combined before the addition of the hydrogen cyanide. In some embodiments, the dinitrile compound, e.g., the dinitrile compound solution, and the hydrogen cyanide may be combined before the addition of the aldehyde.

Mixtures, e.g., solutions, formed by the combining of two or more reactants, e.g., the first, second, or third intermediate solutions, may be referred to as reaction mixtures

In some embodiments, the pH of one or more reaction mixtures is controlled during the reaction. As noted above, the reaction of the present disclosure may be conducted in an acidic environment. In some embodiments, the pH is maintained throughout the reaction. For example, the pH of the reaction may be adjusted, e.g., re-adjusted, after the reactants are combined, because the combination of the reactants may affect the pH of the reaction mixture. In some embodiments, the pH of the reaction mixture may be monitored, e.g., with a pH meter, and a mineral acid may be added to the reaction mixture if the measured pH increases with time.

In some embodiments, the reaction mixture is maintained at a pH ranging from 0.5 to 4, e.g., from 0.5 to 3.8, from 0.5 to 3.6, from 0.5 to 3.4, from 0.5 to 3.2, from 0.5 to 3.0, from 0.8 to 4, from 0.8 to 3.8, from 0.8 to 3.6, from 0.8 to 3.4, from 0.8 to 3.2, from 0.8 to 3.0, from 1.0 to 4, from 1.0 to 3.8, from 1.0 to 3.6, from 1.0 to 3.4, from 1.0 to 3.2, from 1.0 to 3.0, from 1.2 to 4, from 1.2 to 3.8, from 1.2 to 3.6, from 1.2 to 3.4, from 1.2 to 3.2, from 1.2 to 3.0, from 1.5 to 4, from 1.5 to 3.8, from 1.5 to 3.6, from 1.5 to 3.4, from 1.5 to 3.2, or from 1.5 to 3.0.

The temperature of the reaction mixture can also greatly affect the process of the reaction. For example, the temperature of the reaction mixture may affect the solubility of the reactants in solution and/or the rate of the reaction. It is therefore desirable to control the temperature of the reaction mixture. As noted above, for example, each of the reactants, e.g., the dinitrile compound solution, the acetaldehyde, and/or the hydrogen cyanide, may be heated before combining. In some embodiments, the temperature of the reaction mixture may also be controlled, or maintained. The method of controlling the temperature of the reaction mixture is not particularly limited. In some embodiments, a mechanical thermal control, e.g., a heat well or a hot plate, may be used to control the temperature of the reaction mixture.

In some embodiments, the temperature of one or more reaction mixtures is controlled during the reaction. In some embodiments, for example, the mixture of the dinitrile compound, e.g., the dinitrile compound solution, the aldehyde, the hydrogen cyanide, and the nitrile intermediate seed is heated. In some embodiments, the reaction mixture is heated to a temperature from 30° C. and 80° C., e.g., from 30° C. to 75° C., from 30° C. to 70° C., from 30° C. to 65° C., from 30° C. to 60° C., from 32° C. and 80° C., from 32° C. to 75° C., from 32° C. to 70° C., from 32° C. to 65° C., from 32° C. to 60° C., from 35° C. and 80° C., from 35° C. to 75° C., from 35° C. to 70° C., from 35° C. to 65° C., from 35° C. to 60° C., from 38° C. and 80° C., from 38° C. to 75° C., from 38° C. to 70° C., from 38° C. to 65° C., from 38° C. to 60° C., from 40° C. and 80° C., from 40° C. to 75° C., from 40° C. to 70° C., from 40° C. to 65° C., from 40° C. to 60° C., from 42° C. and 80° C., from 42° C. to 75° C., from 42° C. to 70° C., from 42° C. to 65° C., or from 42° C. to 60° C. In terms of lower limits, the reaction mixture may be heated to a temperature greater than 30° C., e.g., greater than 32° C., greater than 35° C., greater than 38° C., greater than 40° C., or greater than 42° C. In terms of upper limits, the reaction mixture may be heated to a temperature less than 80° C., e.g., less than 75° C., less than 70° C., less than 65° C., or less than 60° C. In some cases, the reaction mixture may be heated to a temperature of about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 56° C., about 57° C., about 58° C., about 59° C., about 60° C., about 61° C., about 62° C., about 63° C., about 64° C., or about 65° C.

The rate of heating the reaction mixture is not particularly limited. In one embodiment, for example, the reaction mixture is heated at a rate from 1° C./hour to 60° C./hour, e.g., from 1° C./hour to 50° C./hour, from 1° C./hour to 40° C./hour, from 1° C./hour to 30° C./hour, from 1° C./hour to 20° C./hour, from 3° C./hour to 60° C./hour, from 3° C./hour to 50° C./hour, from 3° C./hour to 40° C./hour, from 3° C./hour to 30° C./hour, from 3° C./hour to 20° C./hour, from 6° C./hour to 60° C./hour, from 6° C./hour to 50° C./hour, from 6° C./hour to 40° C./hour, from 6° C./hour to 30° C./hour, from 6° C./hour to 20° C./hour, from 10° C./hour to 60° C./hour, from 10° C./hour to 50° C./hour, from 10° C./hour to 40° C./hour, from 10° C./hour to 30° C./hour, from 10° C./hour to 20° C./hour, from 10° C./hour to 60° C./hour, from 10° C./hour to 50° C./hour, from 10° C./hour to 40° C./hour, from 10° C./hour to 30° C./hour, or from 10° C./hour to 20° C./hour.

The reaction mixture may be maintained at the heated temperature in order to ensure that the reaction runs to completion. In some cases, the reacting includes maintaining the heated temperature for a period of time. In some embodiments, for example, the heated temperature of the reaction mixture may be maintained for from 30 minutes to 180 minutes, e.g., from 30 minutes to 150 minutes, from 30 minutes to 120 minutes, from 30 minutes to 90 minutes, from 45 minutes to 180 minutes, from 45 minutes to 150 minutes, from 45 minutes to 120 minutes, or from 45 minutes to 90 minutes. In terms of lower limits, the heated temperature may be maintained for at least 30 minutes, at least 35 minutes, at least 40 minutes, at least 45 minutes, at least 50 minutes, or at least 55 minutes. In terms of upper limits, the heated temperature may be maintained for less than 180 minutes, e.g., less than 165 minutes, less than 150 minutes, less than 135 minutes, less than 120 minutes or less than 105 minutes. In some cases, the heated temperature is maintained for about 45 minutes, about 50 minutes, about 55 minutes, about 60 minutes, about 65 minutes, about 70 minutes, about 75 minutes, about 80 minutes, about 85 minutes, or about 90 minutes.

In some embodiments, the reacting comprises some combination of the above-described conditions and parameters. Said another way, the reacting may comprise any combination of the above described temperature, pH, and mixing parameters. In some embodiments, for example, the reacting may include providing a dinitrile compound solution comprising the dinitrile compound, adjusting the pH of the dinitrile compound solution to a pH ranging from 0.5 to 4.0, heating the dinitrile compound solution, adding the aldehyde to the heated dinitrile compound solution to form a first intermediate solution, adding the nitrile intermediate seed to the first intermediate solution to form a second intermediate solution, adding hydrogen cyanide to the second intermediate solution to form a third intermediate solution, and/or heating the third intermediate solution to form the nitrile intermediate.

### Reaction Intermediate

In some cases, the reaction of the dinitrile compound, the aldehyde, and the hydrogen cyanide may produce a reaction intermediate. For example, a reaction intermediate may be produced by a reaction between the dinitrile compound and the aldehyde. In some embodiments, the reaction intermediate may further produce the aforementioned nitrile intermediate. For example, the reaction intermediate may react with the one or more reactants, e.g., the dinitrile compound, the aldehyde, and the hydrogen cyanide, to produce the nitrile intermediate. In some embodiments, the reaction intermediate may react with the solvent, e.g., an aqueous solvent. For example, the reaction intermediate may be hydrolyzed to produce the nitrile intermediate.

The reaction intermediate is not particularly limited and will vary with the reactants, e.g., the dinitrile compound, the aldehyde, and the hydrogen cyanide. Generally, the reaction intermediate is an aminonitrile, e.g., an organic compound having at least one amino functional group and at least two nitrile, or cyano, functional groups. In some embodiments, the reaction intermediate is a compound having the chemical structure:

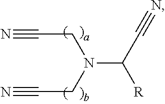

wherein a is from 0 to 5, b is from 0 to 5, and R is (C₁-C₁₀)alkyl, (C₁-C₁₀)haloalkyl, (C₁-C₁₀)alkenyl, or (C₁-C₁₀)alkyl carboxylate. In particular, a and b may correspond to their respective values in the dinitrile compound, and R may correspond to its respective value in the aldehyde. Exemplary reaction intermediates include 1-amino-1-cyanoethane-N,N-diacetonitrile, 1-amino-1-cyanoethane-N,N-dipropionitrile, 1-amino-1-cyanoethane-N,N-dibutyronitrile, 1-amino-1-cyanoethane-N-acetonitrile-N-propionitrile, 1-amino-1-cyanoethane-N-acetonitrile-N-butyronitrile, 1-amino-1-cyanopropane-N,N-diacetonitrile, 1-amino-1-cyanopropane-N,N-dipropionitrile, 1-amino-1-cyanopropane-N,N-dibutyronitrile, 1-amino-1-cyanopropane-N-acetonitrile-N-propionitrile, 1-amino-1-cyanopropane-N-acetonitrile-N-butyronitrile, 1-amino-1-cyanobutane-N,N-diacetonitrile, 1-amino-1-cyanobutane-N,N-dipropionitrile, 1-amino-1-cyanobutane-N,N-dibutyronitrile, 1-amino-1-cyanobutane-N-acetonitrile-N-propionitrile, and 1-amino-1-cyanobutane-N-acetonitrile-N-butyronitrile.

### Product, Nitrite Intermediate

As discussed above, the reaction of the dinitrile compound, the aldehyde, and the hydrogen cyanide produce a nitrile intermediate. The nitrile intermediate is not particularly limited and will vary with the dinitrile compound. Generally, the nitrile intermediate is an organic compound having at least two nitrile, or cyano, functional groups and at least one carboxyl functional group. In some embodiments, the nitrile intermediate is a compound having the chemical structure:

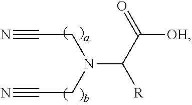

wherein a is from 0 to 5, b is from 0 to 5, and R is (C₁-C₁₀)alkyl, (C₁-C₁₀)haloalkyl, (C₁-C₁₀)alkenyl, or (C₁-C₁₀)alkyl carboxylate. In some embodiments, the nitrile intermediate may have the above chemical structure, wherein a is 1, and b is 0, 1, 2, 3, 4, or 5. In some embodiments, the nitrile intermediate may have the above chemical structure, wherein a is 1 or 2, and be is 0, 1, 2, 3, or 4. In some embodiments, the nitrile intermediate may have the above chemical structure, wherein a is 1, 2, or 3, and b is 1, 2, or 3. In some embodiments, R of the nitrile intermediate is (C₁-C₁₀)alkyl, e.g., (C₁-C₉)alkyl, (C₁-C₈)alkyl, (C₁-C₇)alkyl, (C₁-C₆)alkyl, or (C₁-C₅)alkyl. In some embodiments, R of the nitrile intermediate is (C₁-C₁₀)haloalkyl, e.g., (C₁-C₉)haloalkyl, (C₁-C₈)haloalkyl, (C₁-C₇)haloalkyl, (C₁-C₆)haloalkyl, or (C₁-C₅)haloalkyl. In some embodiments, R of the nitrile intermediate is (C₁-C₁₀)alkenyl, e.g., (C₂-C₁₀)alkenyl, (C₁-C₉)alkenyl, (C₂-C₉)alkenyl, (C₁-C₈)alkenyl, (C₂-C₈)alkenyl, (C₁-C₇)alkenyl, (C₂-C₇)alkenyl, (C₁-C₆)alkenyl, (C₂-C₆)alkenyl, (C₁-C₅)alkenyl, or (C₂-C₅)alkenyl. In some embodiments, R of the nitrile intermediate is (C₁-C₁₀)alkyl carboxylate, e.g., (C₁-C₉)alkyl carboxylate, (C₁-C₈)alkyl carboxylate, (C₁-C₇)alkyl carboxylate, (C₁-C₆)alkyl carboxylate, or (C₁-C₅)alkyl carboxylate. In particular, a and b may correspond to their respective values in the dinitrile compound, and R may correspond to its respective value in the aldehyde.

Exemplary nitrile intermediates include alanine-N,N-diacetonitrile, alanine-N,N-dipropionitrile, alanine-N,N-dibutyronitrile, alanine-N-acetonitrile-N-propionitrile, alanine-N-acetonitrile-N-butyronitrile, ethyl glycine-N,N-diacetonitrile, ethyl glycine-N,N-dipropionitrile, ethyl glycine-N,N-dibutyronitrile, ethyl glycine-N-acetonitrile-N-propionitrile, ethyl glycine-N-acetonitrile-N-butyronitrile, propyl glycine-N,N-diacetonitrile, propyl glycine-N,N-dipropionitrile, propyl glycine-N,N-dibutyronitrile, propyl glycine-N-acetonitrile-N-propionitrile, and propyl glycine-N-acetonitrile-N-butyronitrile.

As has been discussed, the processes described herein produce the nitrile intermediate in crystalline form (i.e. crystalline solid). Said another way, crystals of the nitrile intermediate are produced by the described processes, in particular without need for a separate crystallization step. Furthermore, the nitrile intermediate does not form an emulsion and therefore does not require additional mechanical processing, e.g., agitation, to separate. The formation of the nitrile intermediate in crystalline form increases the efficiency of the production process by removing the need for an additional step (and eliminating the time and cost associated therewith).

The process described herein produces the nitrile intermediate at high yield. In some embodiments, the nitrile intermediate is formed at a yield greater than 70%, e.g., greater than 75%, greater than 80%, greater than 85%, greater than 90%. In terms of upper limits, the nitrile intermediate may be formed at a yield less than 100%, e.g., less than 99.9%, less than 99.5%, less than 99%, or less than 98%.

### Further Reaction

As discussed above, the present disclosure also provides reaction pathways that include preparing the glycine derivative, e.g., alanine-N,N-diacetic acid, from the nitrile intermediate formed by the processes described herein, e.g., alanine-N,N-dinitrile. The structure of the glycine derivative is not particularly limited. As its name suggests, the glycine derivative may be a structural derivative of the amino acid glycine. In particular, the glycine derivative may be any organic compound having at least one carboxy functional group and at least one amino functional group, wherein the carboxy functional group and the amino functional group are separated by one carbon atom. In some embodiments, the carbon atom separating carboxy and amino functional groups may be modified with additional moieties. In some embodiments, the nitrogen of the amino functional group may be modified with additional moieties.

In some embodiments, the glycine derivative is an organic compound having two carboxy-containing functional groups as moieties on the nitrogen atom of the amino functional group. For example, the glycine derivative may have a chemical structure:

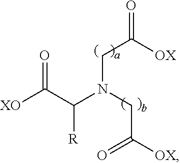

wherein a is from 0 to 5, b is from 0 to 5, and R is (C₁-C₁₀)alkyl, (C₁-C₁₀)haloalkyl, (C₁-C₁₀)alkenyl, or (C₁-C₁₀)alkyl carboxylate. In particular, a and b may correspond to their respective values in the dinitrile compound, and R may correspond to its respective value in the aldehyde. In the above chemical structure, X is hydrogen, an alkali metal, an alkaline earth metal, or ammonium. Exemplary nitrile intermediates include alanine-N,N-diacetic acid, alanine-N,N-dipropionic acid, alanine-N,N-dibutyric acid, alanine-N-acetic acid-N-propionic acid, alanine-N-acetic acid-N-butyric acid, ethyl glycine-N,N-diacetic acid, ethyl glycine-N,N-dipropionic acid, ethyl glycine-N,N-dibutyric acid, ethyl glycine-N-acetic acid-N-propionic acid, ethyl glycine-N-acetic acid-N-butyric acid, propyl glycine-N,N-diacetic acid, propyl glycine-N,N-dipropionic acid, propyl glycine-N,N-dibutyric acid, propyl glycine-N-acetic acid-N-propionic acid, and propyl glycine-N-acetic acid-N-butyric acid.

In the processes described herein, the glycine derivative may be formed by converting the nitrile functional groups of the nitrile intermediate to carboxy functional groups. In particular, the glycine derivative may be formed by hydrolyzing the nitrile intermediate.

Hydrolysis of the nitrile intermediate is not particularly limited and any known method may be used. In some embodiments, the hydrolysis is carried out in an aqueous solution using a strong acid. In some embodiments, the hydrolysis is carried out in an aqueous solution using a strong base. Suitable strong bases include inorganic bases, such as ammonium hydroxide, calcium hydroxide, lithium hydroxide, magnesium hydroxide, potassium hydroxide, sodium hydroxide, and combinations thereof.

The hydrolysis produces the glycine derivative at high yield. In some embodiments, the glycine derivative is formed at a yield greater than 60%, e.g., greater than 65%, greater than 70%, greater than 85%, greater than 90%. In terms of upper limits, the glycine derivative may be formed at a yield less than 100%, e.g., less than 99%, less than 98%, or less than 95%.

It will be appreciated that variants of the above-disclosed and other features and functions, or alternatives thereof, may be combined into many other different systems or applications. Various presently unforeseen or unanticipated alternatives, modifications, variations or improvements therein may be subsequently made by those skilled in the art which are also intended to be encompassed by the following claims or the equivalents thereof.

### Embodiments

As used below, any reference to a series of embodiments is to be understood as a reference to each of those embodiments disjunctively. For example, “Embodiments 1-4” is to be understood as “Embodiments 1, 2, 3, or 4”.

Embodiment 1 is a process for preparing a nitrile intermediate, the process comprising: reacting a dinitrile compound with a hydrogen cyanide and an aldehyde of the formula R—CHO, where R is (C₁-C₁₀)alkyl, (C₁-C₁₀)haloalkyl, (C₁-C₁₀)alkenyl, or (C₁-C₁₀)alkyl carboxylate, in an aqueous solution to form the nitrile intermediate; wherein a nitrile intermediate seed is employed during the reaction.

Embodiment 2 is the process of embodiment(s) 1, wherein the reacting forms the nitrile intermediate as a crystalline solid.

Embodiment 3 is the process of any of the preceding embodiment(s), wherein the process does not comprise a crystallization step.

Embodiment 4 is the process of any of the preceding embodiment(s), wherein the dinitrile compound has a chemical structure:

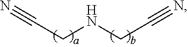

wherein a is from 0 to 5 and b is from 0 to 5.

Embodiment 5 is the process of any of the preceding embodiment(s), wherein the nitrile intermediate is an alanine-N,N-dinitrile.

Embodiment 6 is the process of any of the preceding embodiment(s), wherein the reacting does not form the nitrile intermediate as an emulsion.

Embodiment 7 is the process of any of the preceding embodiment(s), wherein the nitrile intermediate is formed at a yield greater than 70%.

Embodiment 8 is the process of any of the preceding embodiment(s), wherein the reacting comprises providing a dinitrile compound solution comprising the dinitrile compound.

Embodiment 9 is the process of embodiment(s) 8, wherein the reacting comprises heating the dinitrile compound solution to a temperature from 25° C. to 50° C.

Embodiment 10 is the process of embodiment(s) 8, wherein the reacting comprises adjusting the pH of the dinitrile compound solution to a pH ranging from 0.5 to 4.0.

Embodiment 11 is the process of any of the preceding embodiment(s), wherein the reacting comprises adjusting the pH of a reaction mixture to a pH ranging from 0.5 to 4.0.

Embodiment 12 is the process of any of the preceding embodiment(s), wherein the reacting comprises adding the nitrile intermediate seed to a reaction mixture.

Embodiment 13 is the process of any of the preceding embodiment(s), wherein the reacting comprises heating a reaction mixture to a temperature from 30° C. to 80° C.

Embodiment 14 is the process of embodiment(s) 13, wherein the reacting comprises maintaining the reaction mixture at the temperature for a period of time from 30 minutes to 180 minutes.

Embodiment 15 is the process of any of the preceding embodiment(s), wherein the reacting comprises: providing a dinitrile compound solution comprising the dinitrile compound; adjusting the pH of the dinitrile compound solution to a pH ranging from 0.5 to 4.0; heating the dinitrile compound solution; adding the aldehyde to the heated dinitrile compound solution to form a first intermediate solution; adding the nitrile intermediate seed to the first intermediate solution to form a second intermediate solution; adding hydrogen cyanide to the second intermediate solution to form a third intermediate solution; and heating the third intermediate solution to form the nitrile intermediate.

Embodiment 16 is the process of any of the preceding embodiment(s), wherein the pH of the reaction mixture is reduced by at least 2.0, optionally by adding sulfuric acid.

Embodiment 17 is the process of any of the preceding embodiment(s), wherein the reacting is carried out at a pH less than 5.0.

Embodiment 18 is the process of any of the preceding embodiment(s), wherein the third intermediate solution is heated at a rate from 1° C./hour to 60° C./hour.

Embodiment 19 is the process of any of the preceding embodiment(s), wherein the amount of nitrile intermediate seed added is less than 1% the theoretical yield of the nitrile intermediate.

Embodiment 20 is the process of any of the preceding embodiment(s), wherein the second intermediate solution comprises from 0.001 wt. % to 1 wt. % nitrile intermediate seed.

Embodiment 21 is the process of any of the preceding embodiment(s), wherein hydrogen cyanide is added at a rate less than 1 g/minute.

Embodiment 22 is the process of any of the preceding embodiment(s), wherein the nitrile intermediate is formed at a yield greater than 90%.

Embodiment 23 is the process of any of the preceding embodiment(s), wherein the reacting is carried out at a temperature from 25° C. to 40° C.

Embodiment 24 is the process of any of the preceding embodiment(s), wherein R is (C₁-C₅)alkyl, a is from 1 to 3, and/or b is from 1 to 3.

Embodiment 25 is the process of any of the preceding embodiment(s), further comprising forming a glycine derivative from the nitrile intermediate.

Embodiment 26 is the process of embodiment(s) 25, wherein the glycine derivative has a chemical structure:

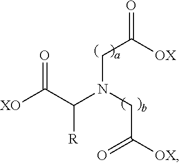

wherein: R is (C₁-C₁₀)alkyl, (C₁-C₁₀)haloalkyl, (C₁-C₁₀)alkenyl, or (C₁-C₁₀)alkyl carboxylate, X is hydrogen, an alkali metal, an alkaline earth metal, or ammonium, a is from 0 to 5, and b is from 0 to 5.

Embodiment 27 is the process of embodiment(s) 25 or claim 26, wherein the forming the glycine derivative comprises hydrolyzing the nitrile intermediate.

Embodiment 28 is the process of any of the embodiment(s) 25-27, wherein the hydrolyzing comprises reacting the nitrile intermediate with an inorganic hydroxide selected from the group consisting of ammonium hydroxide, calcium hydroxide, lithium hydroxide, magnesium hydroxide, potassium hydroxide, sodium hydroxide, and combinations thereof.

Embodiment 29 is the process of any one of embodiment(s)s 25-27, wherein the glycine derivative is alanine-N,N-diacetic acid.

Embodiment 30 is the process of any one of embodiment(s)s 25-28, wherein the glycine derivative is formed at a yield of at least 60%.

### EXAMPLES

The present disclosure will be further understood by reference to the following examples.

### Comparative Example A

((Cyanomethyl)amino)acetonitrile (13.3 g) was added to 50 mL of deionized water at room temperature to produce a dinitrile compound solution. Acetaldehyde (7.8 g) was added to the dinitrile compound solution. The resulting solution was heated to 25° C. Sulfuric acid was added to adjust the pH to 2.0-2.5. Hydrogen cyanide (4.9 g) was then added over half an hour. While the hydrogen cyanide was being added, the solution was heated to 50° C. After the addition of the hydrogen cyanide was complete, the solution was maintained at 50° C. and stirred for three hours.

After three hours, a seed of alanine-N,N-dinitrile (<0.5 g) was added. The solution was the chilled to 5° C. and maintained at 5° C. for one hour. After one hour, the solution was a cloudy emulsion, and no crystal products were observed. The solution was left at room temperature for one week, and the solution remained a cloudy emulsion. No crystals were observed, and the yield was not calculable.

### Example 1

((Cyanomethyl)amino)acetonitrile (13.3 g) was added to 70 mL of deionized water at room temperature to produce a dinitrile compound solution. Sulfuric acid was added to the dinitrile compound solution so as to adjust the pH of the dinitrile compound solution from 7.9 to 2.0. The acidified dinitrile compound solution was then heated to 30-35° C. Acetaldehyde (7.8 g) was added to the dinitrile compound solution to form a first intermediate solution. A seed of alanine-N,N-dinitrile (0.031 g) was added to the first intermediate solution to form a second intermediate solution. Hydrogen cyanide (4.9 g) was then added to the second intermediate solution over an hour to form a third intermediate solution. While the hydrogen cyanide was being added, the third intermediate solution was heated to 50° C. After the addition of the hydrogen cyanide was complete, the third intermediate solution was maintained at 50° C. and stirred for one hour.

After an hour, the third intermediate solution was cooled to room temperature. While the solution cooled, crystalline alanine-N,N-dinitrile (nitrile intermediate) formed. The solid crystals were filtered and dried overnight. After drying, the crystals of alanine-N,N-dinitrile weighed 19.4 g, corresponding to a crude yield of 92%.

As noted, the experiment of Comparative Example A did not add a nitrile intermediate seed during the reaction, and the experiment of Example 1 did. As a result, the reaction of Example 1 readily produced alanine-N,N-dinitrile product as crystals, while the reaction of Comparative Example A produced a cloudy solution without crystals. Because Comparative Example A did not produce crystals, additional processing, e.g., specialized agitations and/or recrystallization methods, are required to collect the nitrile intermediate product. The crystals of Example 1, meanwhile, could be collected simply, e.g., by filtration.

We claim:

1. A process for preparing a nitrile intermediate, the process comprising:
reacting a dinitrile compound with a hydrogen cyanide and an aldehyde of the formula R—CHO, where R is (C₁-C₁₀)alkyl, (C₁-C₁₀)haloalkyl, (C₁-C₁₀)alkenyl, or (C₁-C₁₀)alkyl carboxylate, in an aqueous solution to form the nitrile intermediate;
wherein a nitrile intermediate seed is employed during the reaction.

2. The process of claim 1, wherein the nitrile intermediate is a crystalline solid.

3. The process of claim 1, wherein the dinitrile compound has a chemical structure: 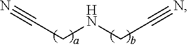
wherein a is from 0 to 5 and b is from 0 to 5.

4. The process of claim 1, wherein the nitrile intermediate is an alanine-N,N-dinitrile.

5. The process of claim 1, wherein the nitrile intermediate seed is added during the reaction.

6. The process of claim 1, wherein the reaction further comprises:
providing a dinitrile compound solution comprising the dinitrile compound;
adjusting the pH of the dinitrile compound solution to a pH ranging from 0.5 to 4.0;
heating the dinitrile compound solution;
adding the aldehyde to the heated dinitrile compound solution to form a first intermediate solution;
adding the nitrile intermediate seed to the first intermediate solution to form a second intermediate solution;
adding hydrogen cyanide to the second intermediate solution to form a third intermediate solution; and
heating the third intermediate solution to form the nitrile intermediate.

7. The process of claim 6, wherein the dinitrile compound solution is heated to a temperature from 25° C. to 50° C.

8. The process of claim 6, wherein the third intermediate solution is heated at a rate from 1° C./hour to 60° C./hour.

9. The process of claim 6, wherein the amount of nitrile intermediate seed added is less than 1% the theoretical yield of the nitrile intermediate.

10. The process of claim 6, wherein the second intermediate solution comprises from 0.001 wt. % to 1 wt. % nitrile intermediate seed.

11. The process of claim 6, wherein hydrogen cyanide is added at a rate less than 1 g/minute.

12. The process of claim 1, wherein the reaction is carried out at a pH less than 5.0.

13. The process of claim 1, wherein the reaction is carried out at a temperature from 25° C. to 40° C.

14. The process of claim 1, further comprising forming a glycine derivative from the nitrile intermediate.

15. The process of claim 14, wherein the glycine derivative has a chemical structure: 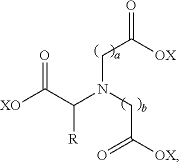
wherein:
R is (C₁-C₁₀)alkyl, (C₁-C₁₀)haloalkyl, (C₁-C₁₀)alkenyl, or (C₁-C₁₀)alkyl carboxylate,
X is hydrogen, an alkali metal, an alkaline earth metal, or ammonium,
a is from 0 to 5, and
b is from 0 to 5.

16. The process of claim 14, wherein R is (C₁-C₅)alkyl, a is from 1 to 3, and/or b is from 1 to 3.

17. The process of claim 14, wherein the forming the glycine derivative comprises hydrolyzing the nitrile intermediate.

18. The process of claim 17, wherein the hydrolyzing comprises reacting the nitrile intermediate with an inorganic hydroxide selected from the group consisting of ammonium hydroxide, calcium hydroxide, lithium hydroxide, magnesium hydroxide, potassium hydroxide, sodium hydroxide, and combinations thereof.

19. The process of claim 14, wherein the glycine derivative is alanine-N,N-diacetic acid.

20. The process of claim 14, wherein the glycine derivative is formed at a yield of at least 60%.
